(19) United States
(12) Patent Application Publication
(10) Pub. No.: US 2013/0289640 A1
(43) Pub. Date: Oct. 31, 2013

(54) HEART SOUND-BASED PACING VECTOR SELECTION SYSTEM AND METHOD

(71) Applicants: Xusheng Zhang, Shoreview, MN (US); Jeffrey M. Gillberg, Coon Rapids, MN (US)

(72) Inventors: Xusheng Zhang, Shoreview, MN (US); Jeffrey M. Gillberg, Coon Rapids, MN (US)

(73) Assignee: Medtronic, Inc.

(21) Appl. No.: 13/458,009

(22) Filed: Apr. 27, 2012

(51) Int. Cl. A61N 1/365

(52) U.S. Cl. 607/17

(57) ABSTRACT

A system and method for generating a pacing vector selection table senses a heart sound signal generated by a heart sound sensor and representing sounds generated by the heart of the patient. A processor controls the sequential selection of a pacing electrode vectors from electrodes positioned along a heart chamber. Pacing pulses are delivered via the sequentially selected plurality of pacing electrode vectors. The processor receives the heart sound signal, determines a plurality of different pacing responses using the heart sound signal for each of the of pacing electrode vectors, and generates a pacing vector selection table listing the plurality of different pacing responses for each of the plurality of pacing electrode vectors.

### FIELD OF THE DISCLOSURE

This disclosure relates to medical devices and, more particularly, to medical devices that delivery cardiac pacing therapy.

### BACKGROUND

Cardiac resynchronization therapy (CRT) is a treatment for heart failure patients in which one or more heart chambers are electrically stimulated (paced) to restore or improve heart chamber synchrony. Improved heart chamber synchrony is expected to improve hemodynamic performance of the heart, such as measured by ventricular pressure and the rate of change in ventricular pressure or other hemodynamic measures. Achieving a positive clinical benefit from CRT is dependent on several therapy control parameters, such as the atrioventricular (AV) delay and the ventricular-ventricular (VV) delay. The AV delay controls the timing of ventricular pacing pulses relative to an intrinsic atrial depolarization or atrial pacing pulse. The ventricular-ventricular (VV) delay controls the timing of a pacing pulse in one ventricle relative to a paced or intrinsic sensed event in the other ventricle.

Numerous methods for selecting optimal AV and VV delays for use in controlling CRT pacing pulses have been proposed. For example, clinicians may select an optimal AV or W delay using Doppler echocardiography. Such clinical techniques are time-consuming and require an expert technician to perform.

As multi-polar cardiac pacing leads become commercially available, multiple pacing electrode vectors are available for pacing a chamber of the patient's heart. In addition to selecting optimal timing control parameters, the clinician must also select an optimal pacing vector for delivering CRT. A need remains for a system and method for efficiently determining optimal pacing control parameters, including the pacing vector, for reducing clinician burden in selecting the therapy control parameters and maximizing the benefit of the therapy to the patient.

### BRIEF DESCRIPTION OF THE DRAWINGS

FIG. 1 is a schematic diagram of one embodiment of an implantable medical device (IMD) system in which techniques disclosed herein may be implemented to provide therapy to a patient.

FIG. 2 is a block diagram illustrating one example configuration of the IMD shown in FIG. 1.

FIG. 3 is a flow chart of a method for automatically generating a pacing vector look-up table for guiding selection of a pacing vector for therapy delivery.

FIG. 4 is a flow chart of a method for detecting phrenic nerve stimulation (PNS) using a heart sound (HS) signal according to one embodiment.

FIG. 5 is a flow chart of a method for verifying cardiac capture using a HS signal according to one embodiment.

FIG. 6 is a flow chart of a method for selecting an optimal pacing vector and optimizing pacing therapy timing parameters using a HS signal according to one embodiment.

### DETAILED DESCRIPTION

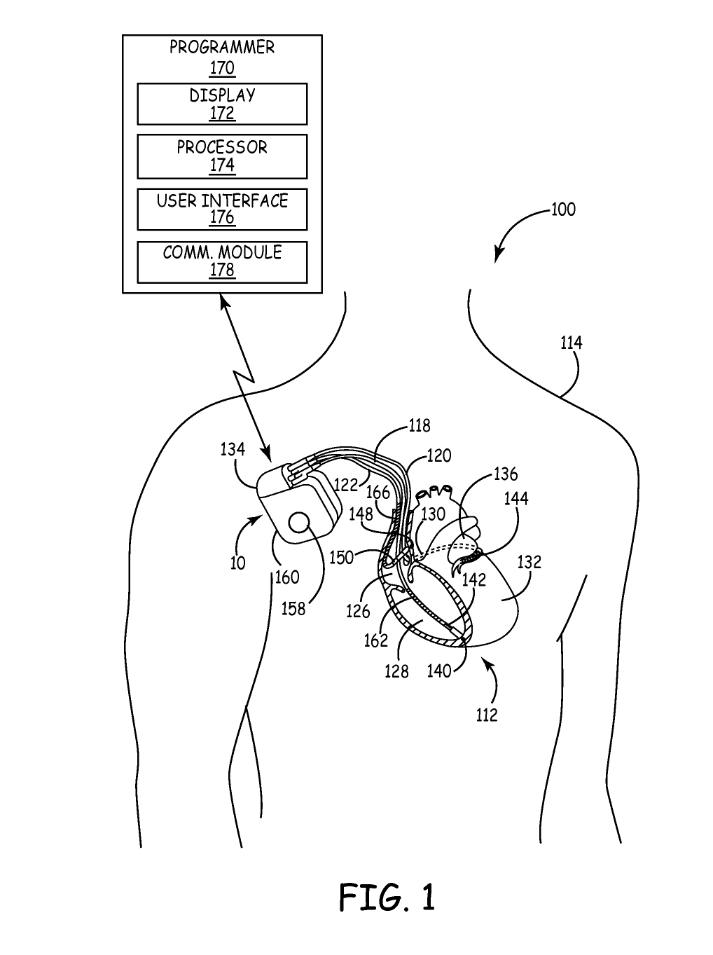

FIG. 1 is a schematic diagram of one embodiment of an implantable medical device (IMD) system 100 in which techniques disclosed herein may be implemented to provide therapy to heart 112 of patient 114. System 100 includes IMD 10 coupled to leads 118, 120, and 122 which carry multiple electrodes. IMD 10 is configured for bidirectional communication with programmer 170. IMD 10 may be, for example, an implantable pacemaker or implantable cardioverter defibrillator (ICD) that provides electrical signals to heart 112 via electrodes coupled to one or more of leads 118, 120, and 122 for pacing, cardioverting and defibrillating the heart 112. IMD 10 is capable of delivering CRT, which may include adaptive CRT which delivers either biventricular or LV-only pacing as needed based on measurements of the patient's intrinsic AV conduction status. In the embodiment shown, IMD 10 is configured for multi-chamber pacing and sensing in the right atrium (RA) 126, the right ventricle (RV) 128, and the left ventricle (LV) 132 using leads 118, 120 and 122.

IMD 10 delivers RV pacing pulses and senses RV intracardiac electrogram (EGM) signals using RV tip electrode 140 and RV ring electrode 142. RV lead 118 is shown carrying a coil electrode 162 which may be used for delivering high voltage cardioversion or defibrillation shock pulses. IMD 10 senses LV EGM signals and delivers LV pacing pulses using the electrodes 144 carried by a multipolar coronary sinus lead 120, extending through the RA 126 and into a cardiac vein 130 via the coronary sinus. In some embodiments, coronary sinus lead 120 may include electrodes positioned along the left atrium (LA) 136 for sensing left atrial EGM signals and delivering LA pacing pulses.

IMD 10 senses RA EGM signals and delivers RA pacing pulses using RA lead 122, carrying tip electrode 148 and ring electrode 150. RA lead 122 is shown to be carrying coil electrode 166 which may be positioned along the superior vena cava (SVC) for use in delivering cardioversion/defibrillation shocks. In other embodiments, RV lead 118 carries both the RV coil electrode 162 and the SVC coil electrode 166. IMD 10 may detect tachyarrhythmias of heart 112, such as fibrillation of ventricles 128 and 132, and deliver cardioversion or defibrillation therapy to heart 112 in the form of electrical shock pulses. While IMD 10 is shown in a right pectoral implant position in FIG. 1, a more typical implant position, particularly when IMD 10 is embodied as an ICD, is a left pectoral implant position.

IMD 10 includes internal circuitry for performing the functions attributed to IMD 10, and a housing 160 encloses the internal circuitry. It is recognized that the housing 160 or portions thereof may be configured as an active electrode 158 for use in cardioversion/defibrillation shock delivery or used as an indifferent electrode for unipolar pacing or sensing configurations. IMD 10 includes a connector block 134 having connector bores for receiving proximal lead connectors of leads 118, 120 and 122. Electrical connection of electrodes carried by leads 118, 120 and 122 and IMD internal circuitry is achieved via various connectors and electrical feedthroughs included in connector block 134.

IMD 10 is configured for delivering CRT therapy, which includes the use of a selected pacing vector for LV pacing that utilizes at least one electrode 144 on multipolar LV lead 120 for unipolar pacing or two of electrodes 144 for bipolar pacing. IMD 10 is configured to pace in one or both ventricles 128 and 132 for controlling and improving ventricular synchrony. The methods described herein can be implemented in a pacemaker or ICD delivering pacing pulses to the right and left ventricles using programmable pacing pulse timing parameters and selected pacing vectors.

Programmer 170 includes a display 172, a processor 174, a user interface 176, and a communication module 178 including wireless telemetry circuitry for communication with IMD 10. In some examples, programmer 170 may be a handheld device or a microprocessor-based home monitor or bedside programming device. A user, such as a physician, technician, nurse or other clinician, may interact with programmer 170 to communicate with IMD 10. For example, the user may interact with programmer 170 via user interface 176 to retrieve currently programmed operating parameters, physiological data collected by IMD 10, or device-related diagnostic information from IMD 10. A user may also interact with programmer 170 to program IMD 10, e.g., select values for operating parameters of the IMD. A user interacting with programmer 170 may request IMD 10 to perform a CRT optimization algorithm for selecting optimal pacing control parameters, which may include pacing vector selection, and transmit results to programmer 170 or request data stored by IMD 10 relating to CRT optimization procedures including pacing vector selection performed automatically by IMD 10 on a periodic basis.

In some embodiments, signal data acquired by the IMD may be transmitted to programmer 170 and programmer 170 performs the CRT optimization algorithm and pacing vector selection using the transmitted signals. The optimization results, i.e. the optimal control parameters and vector selection, would then be transmitted back to the IMD 10 for use in controlling and delivering CRT.

In particular, IMD 10 is configured to generate a table of pacing responses for each of a plurality of pacing electrode vectors for use in selecting an optimal pacing vector. In one embodiment, IMD 10 sequentially selects different unipolar and/or bipolar pacing vectors using one or more of electrodes 144 carried by quadripolar lead 120 and measures a plurality of pacing responses for each pacing vector at one or more pacing pulse energies. It is recognized that in other embodiments, lead 120 may carry a different number of electrodes than the four electrodes shown and thus the number of possible electrode vectors for delivering CRT in a given heart chamber may vary between embodiments.

The pacing responses are used to generate a look-up table of data relied on for selecting an optimal pacing vector for therapy delivery. The pacing responses are determined using heart sound signal analysis as will be described below. The look-up table may be stored in IMD 10 and used to automatically select a pacing vector and/or transferred to programmer 170 for display on display 172 for review by a user, enabling a user to program a pacing vector selection.

Programmer 170 includes a communication module 178 to enable wireless communication with IMD 10. Examples of communication techniques used by system 100 include low frequency or radiofrequency (RF) telemetry, which may be an RF link established via Bluetooth, WiFi, or MICS. In some examples, programmer 170 may include a programming head that is placed proximate to the patient's body near the IMD 10 implant site, and in other examples programmer 170 and IMD 10 may be configured to communicate using a distance telemetry algorithm and circuitry that does not require the use of a programming head and does not require user intervention to establish and/or maintain a communication link.

It is contemplated that programmer 170 may be coupled to a communications network via communications module 178 for transferring data to a remote database or computer to allow remote monitoring and management of patient 114 using the techniques described herein. Remote patient management systems may be configured to utilize the presently disclosed techniques to enable a clinician to review a pacing vector selection look-up table generated using heart sound signal analysis and authorize programming of IMD pacing control parameters, including pacing vector selection. For general descriptions and examples of network communication systems for use with implantable medical devices for remote patient monitoring and device programming, reference is made to commonly-assigned U.S. Pat. No. 6,599,250 (Webb et al.), U.S. Pat. No. 6,442,433 (Linberg et al.), U.S. Pat. No. 6,418,346 (Nelson et al.), and U.S. Pat. No. 6,480,745 (Nelson et al.), all of which patents are hereby incorporated herein by reference in their entirety.

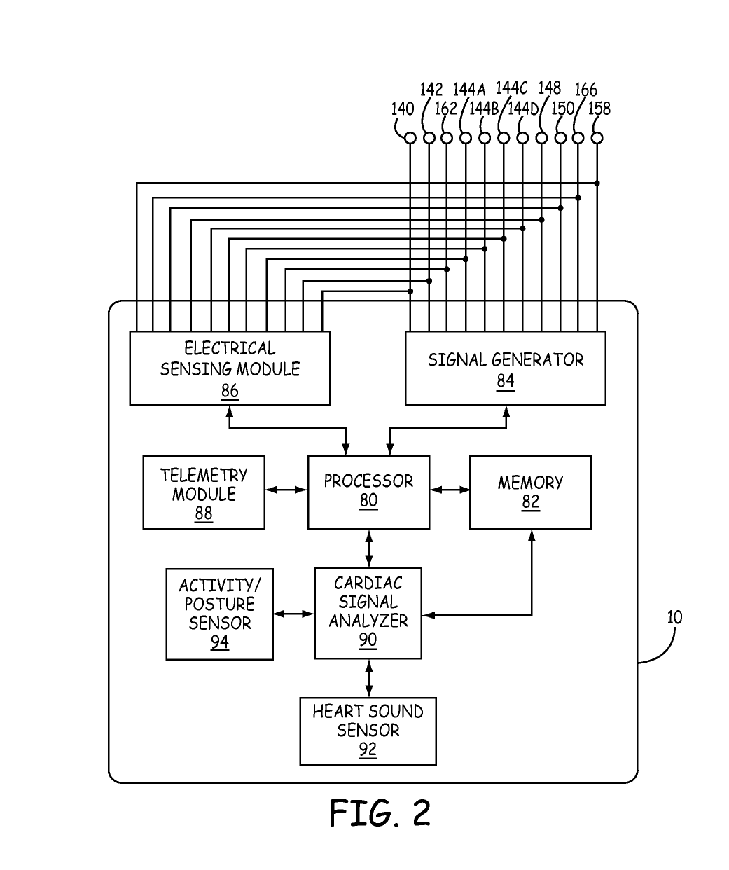

FIG. 2 is a block diagram illustrating one example configuration of IMD 10. In the example illustrated by FIG. 2, IMD 10 includes a processor and control unit 80, also referred to herein as “processor 80”, memory 82, signal generator 84, electrical (EGM) sensing module 86, and telemetry module 88. IMD 10 further includes cardiac signal analyzer 90, heart sound sensor 92 and activity/posture sensor 94.

Memory 82 may include computer-readable instructions that, when executed by processor 80, cause IMD 10 and processor 80 to perform various functions attributed throughout this disclosure to IMD 10, processor 80, and cardiac signal analyzer 90. The computer-readable instructions may be encoded within memory 82. Memory 82 may comprise computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processor and control unit 80 may include any one or more of a microprocessor, a controller, a digital signal processor (DSP), an application specific integrated circuit (ASIC), a field-programmable gate array (FPGA), or equivalent discrete or integrated logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 herein may be embodied as software, firmware, hardware or any combination thereof. In one example, cardiac signal analyzer 90 may, at least in part, be stored or encoded as instructions in memory 82 that are executed by processor and control unit 80.

Processor and control unit 80 includes a therapy control unit that controls signal generator 84 to deliver electrical stimulation therapy, e.g., cardiac pacing or CRT, to heart 112 according to a selected one or more therapy programs, which may be stored in memory 82. Signal generator 84 is electrically coupled to electrodes 140, 142, 144A-144D (collectively 144), 148, 150, 158, 162, and 166 (all of which are shown in FIG. 1), e.g., via conductors of the respective leads 118, 120, 122, or, in the case of housing electrode 158, via an electrical conductor disposed within housing 160 of IMD 10. Signal generator 84 is configured to generate and deliver electrical stimulation therapy to heart 112 via selected combinations of electrodes 140, 142, 144, 148, 150, 158, 162, and 166. Signal generator 84 delivers cardiac pacing pulses according to AV and/or W delays during CRT. These delays are set based on an analysis of cardiac signals by analyzer 90 as will be described herein.

Signal generator 84 may include a switch module (not shown) and processor and control unit 80 may use the switch module to select, e.g., via a data/address bus, which of the available electrodes are used to deliver pacing pulses. Processor 80 controls which of electrodes 140, 142, 144A-144D, 148, 150, 158, 162, and 166 is coupled to signal generator 84 for delivering stimulus pulses, e.g., via the switch module. The switch module may include a switch array, switch matrix, multiplexer, or any other type of switching device suitable to selectively couple a signal to selected electrodes.

During an optimization process, processor 80 sequentially selects different pacing vectors and controls signal generator 84 to vary the pacing pulse energy delivered to a selected pacing vector. Processor 80 generates a look-up table of data including a plurality of different pacing responses corresponding to each pacing vector for one or more pacing pulse energies. The plurality of different pacing responses are measured for each pacing vector (e.g., for a given pacing pulse energy) by cardiac signal analyzer 90 using a signal from heart sound sensor 92. The electrical sensing module 86 may provide signals corresponding to sensed electrical events and/or digitized EGM signals used by cardiac signal analyzer in measuring pacing responses for each pacing vector. A signal from activity/posture sensor 94 may be used by processor 80 in determining when the pacing vector optimization process is performed. As will be described below, the plurality of different pacing responses may include the presence or absence of extra-cardiac stimulation (e.g., phrenic nerve stimulation (PNS)), the pacing capture threshold, and a heart-sound based hemodynamic metric of cardiac function.

Sensing module 86 monitors cardiac electrical signals for sensing cardiac electrical events from selected ones of electrodes 140, 142, 144A-144D, 148, 150, 158, 162, or 166 in order to monitor electrical activity of heart 112. Sensing module 86 may also include a switch module to select which of the available electrodes are used to sense the cardiac electrical activity. In some examples, processor 80 selects the electrodes to function as sense electrodes, or the sensing vector, via the switch module within sensing module 86.

Sensing module 86 includes multiple sensing channels, each of which may be selectively coupled to respective combinations of electrodes 140, 142, 144A-144D, 148, 150, 158, 162, or 166 to detect electrical activity of a particular chamber of heart 112. Each sensing channel may comprise an amplifier that outputs an indication of a sensed event to processor 80 in response to sensing of a cardiac depolarization, in the respective chamber of heart 112. In this manner, processor 80 may receive sensed event signals corresponding to the occurrence of R-waves and P-waves in the various chambers of heart 112. Sensing module 86 may further include digital signal processing circuitry for providing processor 80 or cardiac signal analyzer 90 with digitized EGM signals.

When IMD 10 is configured to deliver adaptive CRT, the occurrence of R-waves in the ventricles, e.g. in the RV, is used in monitoring AV intrinsic conduction time. In particular, prolongation of the AV conduction time or the detection of AV block based on R-wave sensing during no ventricular pacing (or pacing at an extended AV delay that allows intrinsic conduction to take place) is used to control adaptive CRT. When AV conduction is impaired, signal generator 84 is controlled by processor 80 to deliver biventricular pacing, i.e. pacing pulses are delivered in the RV and the LV using a selected AV delay and a selected W delay. When AV conduction is intact, signal generator 84 is controlled by processor 80 to deliver LV-only pacing at a selected AV delay to improve ventricular synchrony.

Memory 82 stores intervals, counters, or other data used by processor 80 to control the delivery of pacing pulses by signal generator 84. Such data may include intervals and counters used by processor 80 to control the delivery of pacing pulses to one or both of the left and right ventricles for CRT. The intervals and/or counters are, in some examples, used by processor 80 to control the timing of delivery of pacing pulses relative to an intrinsic or paced event in another chamber.

Cardiac signal analyzer 90 receives signals from heart sound sensor 92 for determining heart sound-based hemodynamic metrics used to identify optimal CRT control parameters. In addition, the heart sound sensor signal is used to detect other pacing responses, e.g. extra-cardiac capture (e.g., PNS) and/or pacing capture threshold. In alternative embodiments, a different physiological sensor may be used in addition to or substituted for heart sound sensor 92 for providing cardiac signal analyzer 90 with a cardiac signal correlated to cardiac hemodynamic function, particularly ventricular function. Alternative sensors may be embodied as a mechanical, optical or other type of transducer, such as a pressure sensor, oxygen sensor or any other sensor that is responsive to cardiac function and produces a signal corresponding to cardiac mechanical function. Analysis of the heart sound signal is used in guiding selection of the pacing vector and setting optimal AV and VV delays used to control CRT pacing pulses. Cardiac signal analyzer 90 may provide additional EGM signal analysis capabilities using signals from sensing module 86.

Heart sound sensor 92 generates an electrical signal in response to sounds or vibrations produced by heart 112. In addition, the heart sound sensor signal may be responsive to extra-cardiac noise or vibrations, such as the activation of the diaphragm or intercostal muscles due to extra-cardiac capture by the cardiac pacing pulses. Sensor 92 may be implemented as a piezoelectric sensor, a microphone, an accelerometer or other type of acoustic sensor. In some examples, heart sound sensor 92 may be used as both an acoustic to electrical transducer and as an electrical to acoustic transducer. In such examples, the sensor may also be used to generate an audible alarm for the patient, such as a buzzing or beeping noise. The alarm may be provided in response to detecting a hemodynamic metric that crosses an alarm threshold.

In FIG. 2, heart sound sensor 92 is enclosed within housing 160 of IMD 10 with other electronic circuitry. In other examples, heart sound sensor 92 may be formed integrally with or on an outer surface of housing 160 or connector block 134. In still other examples, heart sound sensor 92 is carried by a lead 118, 120, 122 or other lead coupled to IMD 10. In some embodiments, heart sound sensor 92 may be implemented as a remote sensor that communicates wirelessly with IMD 10. In any of these examples, sensor 92 is electrically or wirelessly coupled to cardiac signal analyzer 90 to provide a signal correlated to sounds generated by heart 112 for deriving hemodynamic function metrics and for measuring other responses to CRT delivered using different pacing vectors.

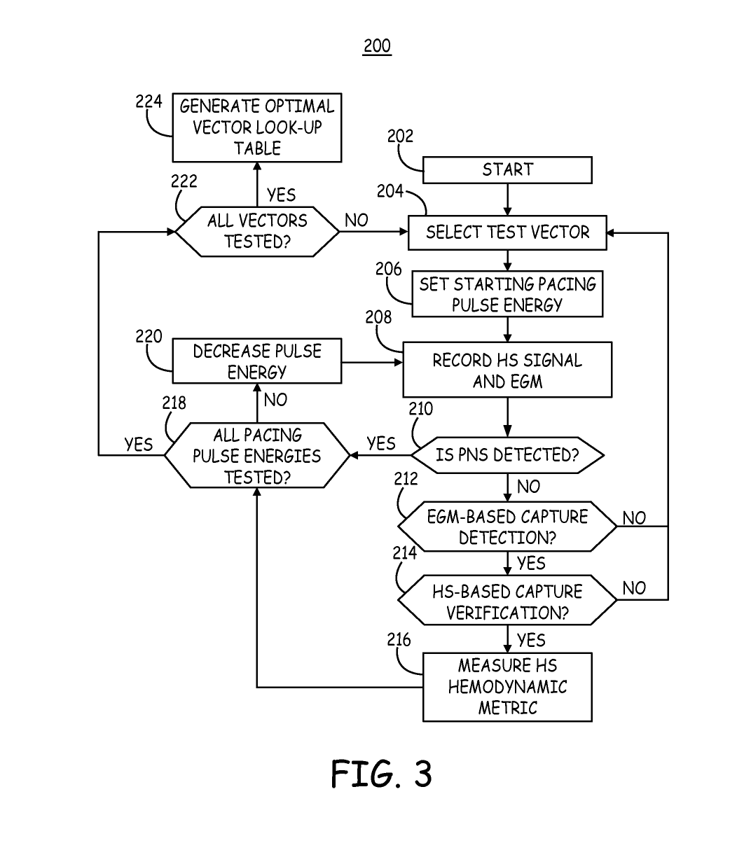

FIG. 3 is a flow chart 200 of a method for automatically generating a pacing vector look-up table for guiding selection of a pacing vector for therapy delivery. Factors considered when selecting which pacing electrode vector to use for pacing a patient's heart may include the pacing capture threshold, the hemodynamic benefit, and the avoidance of extra-cardiac stimulation. When selecting a pacing electrode vector, it is generally desired to avoid selecting an electrode pair that results in relatively high energy expenditure, e.g. due to high pacing capture threshold, in order to avoid early depletion of the IMD battery. Moreover, electrical capture, which can be assessed from the EGM signal, does not necessarily translate to mechanical capture in a sick heart because of possible electromechanical dissociation or delay and mechanical delay, especially in heart failure patients. The actual mechanical response after a pacing pulse, e.g., as evidenced by the existence of heart sounds such as the S1 and/or S2 heart sounds after a pacing pulse, provides a reliable confirmation that pacing has successfully captured the heart to cause a mechanical heart beat. Extra-cardiac capture of the phrenic nerve causing diaphragmatic contraction or of nerves innervating the intercostal muscles may cause the patient discomfort or annoyance. Some pacing vectors may yield greater hemodynamic benefit than other pacing vectors. Each of these aspects may be taken into consideration when selecting a pacing vector for therapy delivery and may therefore be represented in a pacing vector look-up table.

At block 202, a pacing vector testing process is started. The process shown by flow chart 200 may be performed when the IMD system is first implanted, during an office visit, upon a manual trigger, on an automatic periodic basis, or in response to an automatic trigger, for example in response to detecting loss of capture or a worsening of hemodynamic function.

At block 204, a test vector is selected. In the system shown in FIG. 1, the LV lead 120 is embodied as a quadripolar lead. Sixteen pacing vectors are possible using the quadripolar lead. Twelve bipolar pairs can be selected from the four electrodes 144 and each of the four electrodes 144 may be selected one at a time for unipolar pacing, paired with the housing electrode 158, for example. All sixteen pacing vectors may be tested in a sequential manner or a selected subset of the possible vectors. A first pacing vector is selected at block 204 for testing.

A starting pacing pulse energy is automatically selected at block 206 and pacing is delivered, which is LV pacing in this example, using the selected test vector and starting pulse energy. The pacing may be delivered according to a pacing therapy protocol such as a CRT protocol and may therefore be delivered in an LV-only pacing mode or in combination with atrial and/or RV pacing.

A heart sound (HS) signal and an EGM signal are recorded at block 208 during pacing. At block 210, an analysis of the EGM and HS signals is performed to detect the presence of phrenic nerve stimulation (PNS) or more generally any extra-cardiac stimulation. If PNS is detected, and additional pacing pulse energies using the currently selected pacing vector remain to be tested (decision block 218), the processor decreases the pulse energy at block 220 and returns to block 208 to record the HS signal and the EGM signal during pacing at the lower pulse energy. Pacing pulse energy may be reduced by decreasing the pacing pulse signal width and/or reducing the pacing pulse signal amplitude. If PNS is detected at block 210 and all pacing pulse energies to be tested have been applied as determined at block 218 (or at least the lowest pacing pulse energy has been applied), the process advances to block 222 to select the next pacing vector.

If PNS is not detected at block 210, the EGM signal is analyzed to detect electrical capture at block 212. If capture is not detected based on EGM signal

PATENT PNS detection and unsuccessful electrical capture is stored for the current test vector and pacing pulse energy.

If capture is detected based on EGM signal analysis at block 212, the HS signal is analyzed at block 214 to verify mechanical capture detection. If capture is not verified based on HS signal analysis, the next test vector is selected at block 204. If capture is verified, a hemodynamic function metric is derived from the HS signal at block 216 and stored with the corresponding pacing vector and pacing pulse energy.

After measuring the HS-based hemodynamic metric, the processor determines if all pacing pulse energies have been tested for the currently selected pacing vector. If not, the pulse energy is decreased at block 220 and the blocks 208 through 216 are repeated.

If all pacing pulse energies have been applied for the currently selected vector, the processor determines if all pacing vectors to be tested have been selected at block 222. If not, the next test vector is selected at block 204 and the process of analyzing the HS signal and EGM signal for extra-cardiac capture, cardiac capture and deriving a HS-based hemodynamic metric are repeated during pacing at progressively decreasing pacing pulse energies unless PNS is detected or loss of capture occurs. In other embodiments, the pacing pulse energy may start at a low level and be increased or may start at any selected pulse energy and be adjusted in a random, binary search or other pattern.

The pacing vector and pulse energies selected at blocks 204 and 206 respectively may be selected automatically by the processor 80. In some embodiments, a user may enter which pacing vectors should or should not be tested and what pulse energy ranges should or should not be tested, thus having the option to place limits on the tests performed. The pacing vectors and the range of pacing pulse energies to be tested are cycled through automatically under the control of the processor 80.

In some embodiments, the HS-based hemodynamic metric measured at block 216 may be measured at only one pacing pulse energy for a given pacing vector rather than for every pacing pulse energy for which capture is verified. For example, the pulse energy may be progressively decreased until capture is no longer verified based on the HS signal analysis at block 214. The lowest pulse energy at which mechanical capture is verified is stored as the capture threshold for the given pacing vector. A HS-based hemodynamic metric may be derived from the HS signal at block 216 during pacing at a predetermined increment above the cardiac capture threshold and stored as a metric of hemodynamic performance for the given pacing vector.

While the blocks shown in flow chart 200 are shown in a particular order, it is recognized that the various analyses for detecting PNS, EGM-based capture, HS-signal based capture and deriving a HS-based hemodynamic metric may be performed in a different order than the order shown and may be performed in a simultaneous or semi-simultaneous manner rather than in a sequential manner.

As the pacing responses are measured or after all pacing responses for each pacing vector are measured, a pacing vector look-up table is generated at block 224. The pacing vector look-up table stores the results of the PNS analysis, capture verification, and HS hemodynamic metric for each pacing vector, and optionally for each pacing energy applied for a given pacing vector.

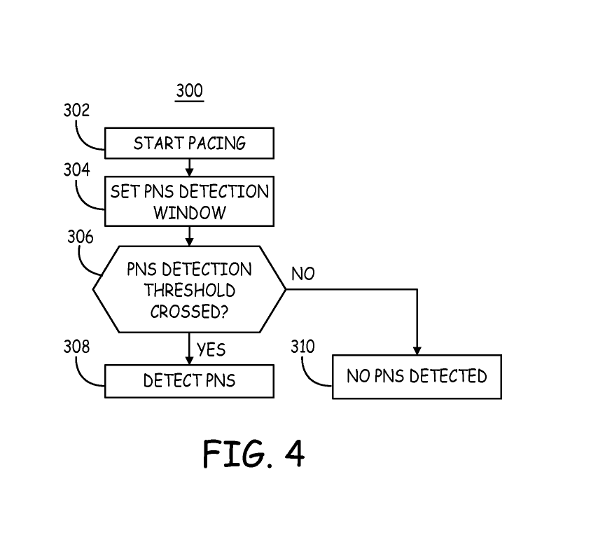

FIG. 4 is a flow chart 300 of a method for detecting PNS using a HS signal according to one embodiment. The pacing therapy is delivered at block 302 using a selected test pacing vector and test pacing pulse energy as described above. A PNS detection window is set at block 304. The window is set as an interval of time beginning at or immediately after the pacing pulse and extending approximately 50 to 100 ms, e.g. approximately 80 ms in one embodiment, after the pacing pulse. The window is set to extend between a pacing pulse and end prior to an expected S1 sound or myocardial depolarization associated with capture of the heart.

During the PNS detection window, the HS signal is recorded and analyzed at block 306 to detect a change in the HS signal indicative of PNS. For example, a determination may be made whether a PNS detection threshold is crossed. In one embodiment, PNS is detected if the HS signal amplitude exceeds an amplitude threshold, which may be a threshold crossing of the filtered HS signal, a threshold crossing of the rectified ensemble-averaged HS signal, a threshold crossing of the peak-to-peak difference (or peak to a baseline) of the HS signal during the PNS detection window. In other embodiments, the PNS detection threshold may include a frequency content criterion for detecting PNS, or more generally detecting capture of non-cardiac excitable tissue.

If the PNS detection threshold is crossed, PNS is detected at block 308. A flag or marker indicating that PNS is detected for the current pacing vector and pulse energy is stored in IMD memory 82. If the PNS detection threshold is not crossed, PNS is not detected at block 310. A flag or marker indicating no PNS may be stored for the associated pacing vector and pulse energy.

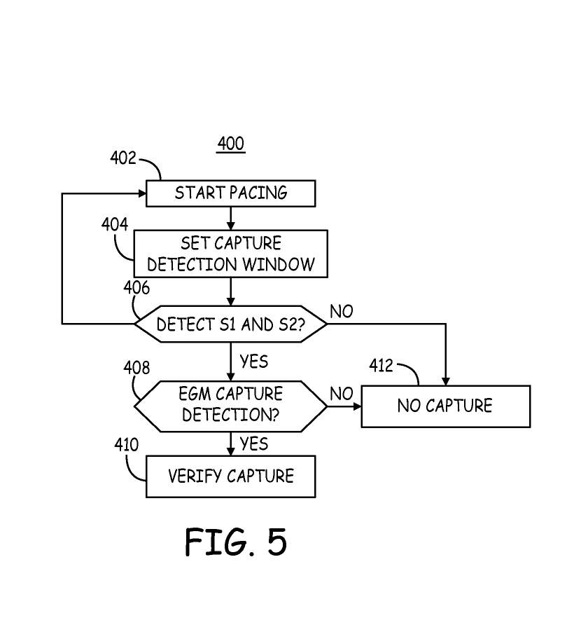

FIG. 5 is a flow chart 400 of a method for verifying cardiac capture using a HS signal according to one embodiment. At block 402, the pacing therapy is delivered using a selected test pacing vector and a selected test pulse energy. At block 404, a capture detection window is set following each pacing pulse. If a change in the HS signal is detected during the cardiac capture detection window, capture is verified. In one embodiment, the capture detection window is applied to the HS signal for detecting whether an S1 and/or S2 signal are present during the cardiac capture detection window at decision block 406. For example, the S1 sound is typically 100-240 ms after ventricular pacing pulse; the S2 sound is typically 370-490 ms after ventricular pacing pulse. A cardiac capture detection window may extend, therefore from approximately 100 ms after a pacing pulse up to approximately 500 ms after the pacing pulse though shorter windows could be used. The length of the cardiac capture detection window may be set based on a current pacing rate and may extend from the end of a PNS detection window.

The S1 and S2 heart sounds can be detected based on a threshold crossing, peak-to-peak amplitude change, signal morphology or other criteria. If the S1 and/or S2 heart sounds are detected, and an EGM-based capture detection is made at decision block 408, capture is verified at block 410. If the S1 and S2 sounds are not detected during the capture detection window at decision block 406, capture is not verified, i.e. loss of capture is detected. If the S1 and/or S2 sound(s) are detected but capture is not detected based on an EGM signal analysis at block 408, loss of capture may still be verified in some embodiments since the EGM signal quality may be compromised. In some embodiments, both the HS signal analysis and the EGM signal analysis are required to result in capture detection in order to verify capture. In other embodiments, the HS signal analysis may be used alone to detect and confirm capture.

If capture is verified at block 410, a flag or marker is stored in memory indicating that the selected pacing vector and pulse energy does result in capture of the heart. If capture is not detected at block 412, a flag or marker is stored in memory indicating that the selected pacing vector and pulse energy fails to capture the heart.

Referring again to FIG. 3, at block 224 an optimal pacing vector look-up table is generated after analyzing the HS signal for PNS detection, capture verification and measuring a hemodynamic metric. A pacing vector look-up table stores an indication of whether PNS was detected, what the mechanical cardiac capture threshold is, and the HS-based hemodynamic measurement for each of the pacing vectors tested. Additionally, the presence or absence of PNS and/or the hemodynamic metric may be stored for multiple pulse energies for a given pacing vector when different pacing pulse energies yield different pacing responses for the given pacing vector. In an alternative embodiment, an extra-cardiac capture threshold may be determined for each pacing vector and stored in the look-up table in an entry corresponding to the pacing vector rather than an indication of PNS presence or absence for each pulse energy.

Table I is an example of one embodiment of an optimal pacing vector look-up table in which values for the cardiac capture threshold, an indication of whether PNS is detected, and a HS-based hemodynamic metric are stored for each test pacing vector. In this example, 16 possible pacing vectors for pacing the LV using a quadripolar lead may be tested. The HS-based hemodynamic parameter is the amplitude of the S1 sound, which is used as a surrogate for LV dP/dt max, which is an indication of LV contractility.

TABLE I
Optimal pacing vector look-up table.

| VECTOR | CAPTURE THRESHOLD | PNS | S1 AMPLITUDE |
|---|---|---|---|
| 1 | mV | No | mV |
| 2 | mv | Yes | mV |
| 3 | mv | No | mV |
| . . . | . . . | . . . | . . . |
| 16 | mV | No | mV |

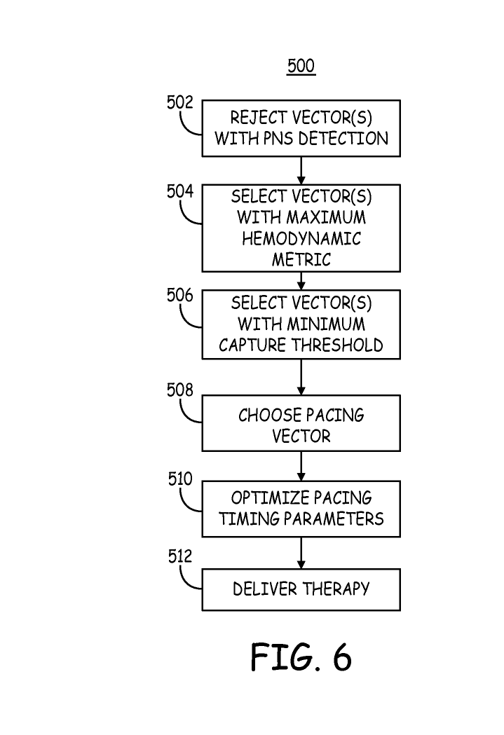

FIG. 6 is a flow chart 500 of a method for selecting an optimal pacing vector and optimizing pacing therapy timing parameters using a HS signal according to one embodiment. At block 502, the look-up table is used to identify any pacing vectors associated with PNS detection. Those pacing vectors are rejected. Of the remaining vectors listed in the look-up table, the pacing vector having a maximum HS-based hemodynamic metric is selected at block 504. If more than one of the remaining vectors is associated with a maximum hemodynamic metric, all vectors having the highest hemodynamic metric (with no PNS detection) are selected at block 504. It is recognized that depending on what the hemodynamic metric is, the best hemodynamic performance may be associated with a minimized HS-based hemodynamic metric in which case the pacing vector(s) associated with a minimized metric or another target value or range are selected at block 504.

Of the vectors selected at block 504, the vector with the lowest pacing capture threshold is selected at block 506. The selected pacing vector is chosen as the therapy delivery pacing vector at block 508. In an alternative method for selecting an optimal pacing vector from the look-up table, vectors associated with PNS are first rejected. Of the remaining vectors, the pacing vectors having the lowest pacing capture threshold, verified by both the EGM (electrical) and HS signal (mechanical) capture detection analysis, are selected. From the vectors having no PNS and lowest electrical and mechanical capture threshold, the vector having a maximum hemodynamic measurement, e.g. maximum S1 amplitude, is selected.

In some cases, more than one vector may meet the selection criteria of having a maximum hemodynamic response and minimum pacing capture with no extra-cardiac capture. If more than one vector remains after applying selection criteria, a nominal one of the remaining vectors may be chosen as the pacing vector at block 508. In some embodiments, the process of choosing the pacing vector at block 508 may include performing a pacing impedance measurement when more than one vector remains. The vector having the highest pacing impedance is selected as the pacing vector for therapy delivery at block 508. A higher pacing impedance will result in lower battery drain and longer battery life.

After choosing the optimal pacing vector, optimization of pacing therapy control parameters is performed at block 510. For example, if the pacing vector is chosen for LV pacing during CRT, an AV delay and/or a VV delay are optimized at block 510 to provide a maximum hemodynamic response using the chosen vector. An AV delay may be optimized for use during LV-only pacing modes, and an AV delay and a VV delay may be optimized for use during biventricular pacing modes. The HS signal may be analyzed and used for determining optimal timing control parameters. Numerous techniques may be used for determining the optimal timing parameters. Reference is made, for example, to U.S. Pat. application Ser. No. 13/111,260, filed May 19, 2011, hereby incorporated herein by reference in its entirety.

The techniques described herein for generating an optimal pacing vector look-up table may be repeated periodically or in response to a change in a monitored HS-based hemodynamic monitor or detecting a loss of capture. Each time a new pacing vector is selected, a timing parameter optimization may be performed to promote maximum patient benefit from the pacing therapy.

Thus, a medical device system and associated methods have been presented in the foregoing description with reference to specific embodiments for using heart sound signals in generating an optimal pacing vector look-up table and choosing a pacing vector for therapy delivery. It is appreciated that various modifications to the referenced embodiments may be made without departing from the scope of the disclosure as set forth in the following claims. For example, any of the techniques or processes described in conjunction with block diagrams and flow charts presented herein may be combined or functional blocks may be omitted or re-ordered in alternative embodiments. The description of the embodiments is illustrative in nature and, thus, variations that do not depart from the gist of the disclosure are intended to be within the scope of the disclosure and claims.

1. A method for selecting a pacing therapy electrode vector, comprising:
sensing a heart sound signal generated by a heart sound sensor and representing sounds generated by the heart of the patient;
sequentially selecting a plurality of pacing electrode vectors from a plurality of electrodes positioned along a heart chamber;
delivering pacing pulses via the sequentially selected plurality of pacing electrode vectors;
enabling a processor to receive the heart sound signal, determine a plurality of different pacing responses in response to the heart sound signal for each of the plurality of pacing electrode vectors, and generate a table comprising the plurality of different pacing responses for each of the plurality of pacing electrode vectors.

2. The method of claim 1, further comprising:
setting an extra-cardiac detection window extending between a pacing pulse and an expected myocardial response to the pacing pulse;
detecting a change in the heart sound signal during the extra-cardiac detection window; and
detecting extra-cardiac stimulation in response to the heart sound signal change, wherein the plurality of different pacing responses comprises extra-cardiac stimulation detection.

3. The method of claim 1, further comprising:
setting a cardiac capture detection window;
detecting a change in the heart sound signal during the cardiac capture detection window correlated to myocardial contraction; and
detecting cardiac capture in response to detecting the heart sound signal change, wherein the plurality of pacing responses comprises cardiac capture detection.

4. The method of claim 1, further comprising:
computing a hemodynamic metric from the heart sound signal, wherein the plurality of pacing responses comprises the hemodynamic metric.

5. The method of claim 1, wherein determining the plurality of different pacing responses comprises:
determining a presence of phrenic nerve stimulation;
determining a mechanical cardiac capture threshold; and
determining a hemodynamic metric for each of the plurality of pacing electrode vectors.

6. The method of claim 5, further comprising determining the plurality of different pacing responses for a plurality of the pacing pulse energies for each of the plurality of pacing electrode vectors.

7. The method of claim 1, further comprising generating a display of the look-up table.

8. The method of claim 1, further comprising:
performing a comparative analysis of the different pacing responses; and
identifying an optimal pacing vector in response to the comparative analysis.

9. The method of claim 8, further comprising:
automatically selecting the optimal pacing vector;
delivering cardiac resynchronization therapy using the selected optimal pacing vector;
adjusting a timing parameter for controlling the cardiac resynchronization therapy;
selecting an optimal timing parameter in response to the heart sound signal; and
delivering the cardiac resynchronization therapy using the optimal timing parameter and the optimal pacing vector.

10. The method of claim 8, wherein identifying an optimal pacing vector further comprises performing a lead impedance measurement.

11. A medical device system, comprising:
a plurality of electrodes positioned along a heart chamber of a patient for delivering cardiac pacing pulses;
a heart sound sensor for generating a heart sound signal representative of sounds generated by a heart of a patient;
a processor configured to sequentially select a plurality of pacing electrode vectors from the plurality of electrodes; and
a signal generator controlled by the processor to deliver pacing pulses via the sequentially selected plurality of pacing electrode vectors, wherein the processor is configured to receive the heart sound signal, determine a plurality of different pacing responses in response to the heart sound signal for each of the plurality of pacing electrode vectors, and generate a table comprising the plurality of different pacing responses for each of the plurality of pacing electrode vectors.

12. The system of claim 11, wherein the processor is configured to:
set an extra-cardiac detection window extending between a pacing pulse and an expected myocardial response to the pacing pulse;
detect a change in the heart sound signal during the extra-cardiac detection window; and
detect extra-cardiac stimulation in response to the heart sound signal change, the plurality of pacing responses comprising extra-cardiac stimulation detection.

13. The system of claim 11, wherein the processor is further configured to:
set a cardiac capture detection window;
detect a change in the heart sound signal during the cardiac capture detection window correlated to myocardial contraction; and
detect cardiac capture in response to detecting the heart sound signal change, wherein the plurality of different pacing responses comprises cardiac capture detection.

14. The system of claim 11, wherein the processor is further configured to compute a hemodynamic metric from the heart sound signal, wherein the plurality of different pacing responses comprises the hemodynamic metric.

15. The system of claim 11, wherein determining the plurality of different pacing responses comprises:
determining a presence of phrenic nerve stimulation;
determining a mechanical cardiac capture threshold; and
determining a hemodynamic metric for each of the plurality of pacing electrode vectors.

16. The system of claim 15, wherein the processor is further configured to determine the plurality of different pacing responses for a plurality of the pacing pulse energies for each of the plurality of pacing electrode vectors.

17. The system of claim 11, further comprising a display for generating a display of the look-up table.

18. The system of claim 11, wherein the processor is further configured to perform a comparative analysis of the different pacing responses and identify an optimal pacing vector in response to the comparative analysis.

19. The system of claim 18, wherein the processor is further configured to:
automatically select the optimal pacing vector;
deliver cardiac resynchronization therapy using the selected optimal pacing vector;
adjust a timing parameter for controlling the cardiac resynchronization therapy;
select an optimal timing parameter in response to the heart sound signal; and
deliver the cardiac resynchronization therapy using the optimal timing parameter and the optimal pacing vector.

20. The system of claim 18, wherein identifying an optimal pacing vector comprises performing a lead impedance measurement.

21. A non-transitory computer-readable medium storing instructions which cause a medical device system to perform a method, the method comprising:
sensing a heart sound signal generated by a heart sound sensor and representing sounds generated by the heart of the patient;
sequentially selecting a plurality of pacing electrode vectors from a plurality of electrodes positioned along a heart chamber;
delivering pacing pulses via the sequentially selected plurality of pacing electrode vectors;
determining a plurality of different pacing responses in response to the heart sound signal for each of the plurality of pacing electrode vectors; and
generating a table comprising the plurality of different pacing responses for each of the plurality of pacing electrode vectors.
